(19) United States
(12) Patent Application Publication
(10) Pub. No.: US 2026/0284056 A1
(43) Pub. Date: Sep. 24, 2026

(54) IMMUNO-THERAPEUTIC CHEMICAL COMPOSITIONS AND USES THEREOF

(71) Applicant: Phillip MOHENO, SAN DIEGO, CA (US)

(72) Inventor: Phillip MOHENO, SAN DIEGO, CA (US)

(21) Appl. No.: 19/550,782

(22) Filed: Feb. 26, 2026

Related U.S. Application Data
(63) Continuation of application No. 19/280,031, filed on Jul. 24, 2025, now Pat. No. 12,569,492.
(60) Provisional application No. 63/739,478, filed on Dec. 27, 2024.

(51) Int. Cl. A61K 31/519; A61K 31/51; A61K 31/714; A61K 33/06; A61K 33/30; A61K 45/06

(52) U.S. Cl. CPC A61K 31/519; A61K 31/51; A61K 31/714; A61K 33/06; A61K 33/30; A61K 45/06

(57) ABSTRACT

Disclosed herein are methods for the treatment of cancer and inflammatory-based diseases and disorders, such as coronavirus colds and as a therapy against COVID-19. ImmunoFolate has been shown to reduce the incidents of colds and flus. In one embodiment is a method of treating cancer comprising administration of ImmunoFolate. In another embodiment is a method of treatment inflammatory-based disease and disorders comprising administration of ImmunoFolate.

This patent application is a continuation of U.S. Utility patent application Ser. No. 19/280,031, filed 24 Jul. 2025, which claims the benefit of U.S. Provisional Patent Application 63/739,478 filed 27 Dec. 2024, the specifications of which are hereby incorporated herein by reference.

### BACKGROUND

Previously it has been reported that ImmunoPterin (IP) (Folic Acid, calcium chloride) as a therapeutic treatment against coronavirus colds and as a therapy against COVID-19. ImmunoPterin has been shown to reduce the incidents of colds and flus. ImmunoPterin is also identified as Calcium Folate, and consists of a combination of folic acid and Calcium chloride. However, there remain derivative or similar chemical compositions that have yet to be disclosed that provide additional and/or more effective therapeutic treatments. These similar or derivative chemical compositions and their therapeutic treatments are described within this application.

### BRIEF SUMMARY

Aspects and advantages of the disclosure will be set forth in part in the following description, or may be obvious from the description, or may be learned through practice of the disclosure. Various embodiments provide ophthalmic therapeutic treatment compositions.

In one embodiment of the disclosure, a method of treating cancer comprising administration of a composition comprising of “ImmunoFolate”, is provided, wherein ImmunoFolate comprises.
folic acid,
calcium chloride (CaCl₂)) or hydrates thereof or both, and
at least one of
   magnesium chloride (MgCl₂) or hydrates thereof or both,
   Vitamin B12.

In one embodiment of the disclosure, a method of treating cancer comprising administration of a composition comprising of ImmunoFolate, wherein ImmunoFolate has a greater therapeutic efficacy as compared to administration of Calcium Folate, is provided.

In one embodiment of the disclosure, a method of treating cancer comprising administration of a composition comprising of ImmunoFolate, wherein ImmunoFolate is administered through oral, parenteral, intravenous, subcutaneous, intrathecal, intramuscular, buccal, intranasal, epidural sublingual, pulmonary, local, rectal, or transdermal administration, is provided.

In one embodiment of the disclosure, a method of treating cancer comprising administration of a composition of ImmunoFolate plus zinc, is provided.

In one embodiment of the disclosure, a method of treating cancer comprising administration of a composition of ImmunoFolate plus zinc, wherein ImmunoFolate plus zinc is administered through oral, parenteral, intravenous, subcutaneous, intrathecal, intramuscular, buccal, intranasal, epidural sublingual, pulmonary, local, rectal, or transdermal administration, is provided.

In one embodiment of the disclosure, a method of treating an inflammatory-based disease or disorder in a subject comprising administration of a composition of ImmunoFolate, is provided.

In one embodiment of the disclosure, a method of treating an inflammatory-based disease or disorder in a subject comprising administration of a composition of ImmunoFolate, wherein the ImmunoFolate is administered through oral, parenteral, intravenous, subcutaneous, intrathecal, intramuscular, buccal, intranasal, epidural sublingual, pulmonary, local, rectal, or transdermal administration, is provided.

In one embodiment of the disclosure, a method of treating an inflammatory-based disease or disorder in a subject comprising of administration of a composition comprising of ImmunoFolate plus zinc, is provided.

In one embodiment of the disclosure, a method of treating an inflammatory-based disease or disorder in a subject comprising of administration of a composition comprising of ImmunoFolate plus zinc, wherein the ImmunoFolate plus zinc is administered through oral, parenteral, intravenous, subcutaneous, intrathecal, intramuscular, buccal, intranasal, epidural sublingual, pulmonary, local, rectal, or transdermal administration, is provided.

In one embodiment of the disclosure, a method of treating an inflammatory-based disease or disorder in a subject comprising of administration of a composition comprising of ImmunoFolate plus zinc, wherein the inflammatory-based disease or disorder is arthritis, is provided.

In one embodiment of the disclosure, a method of treating an inflammatory-based disease or disorder in a subject comprising of administration of a composition comprising of ImmunoFolate plus zinc, wherein the inflammatory-based disease or disorder is arthritis, wherein the arthritis is osteoarthritis, rheumatoid arthritis, gout, psoriatic arthritis, lupus, or septic arthritis, is provided.

In one embodiment of the disclosure, a method of treating an inflammatory-based disease or disorder in a subject comprising administration of a composition of ImmunoFolate, wherein the inflammatory-based disease or disorder is arthritis, is provided.

In one embodiment of the disclosure, a method of treating an inflammatory-based disease or disorder in a subject comprising administration of a composition of ImmunoFolate, wherein the inflammatory-based disease or disorder is arthritis, wherein the arthritis is osteoarthritis, rheumatoid arthritis, gout, psoriatic arthritis, lupus, or septic arthritis, is provided.

### BRIEF DESCRIPTION OF THE FIGURES

A full and enabling disclosure of the present disclosure, including the best mode thereof, directed to one of ordinary skill in the art, is set forth in the specification, which makes reference to the appended drawings in, which:

FIG. 1 provides the stomach chemistry that occurs upon oral ingestion of the formulations of the chemical composition; and, the hypothesized active chelates generated.

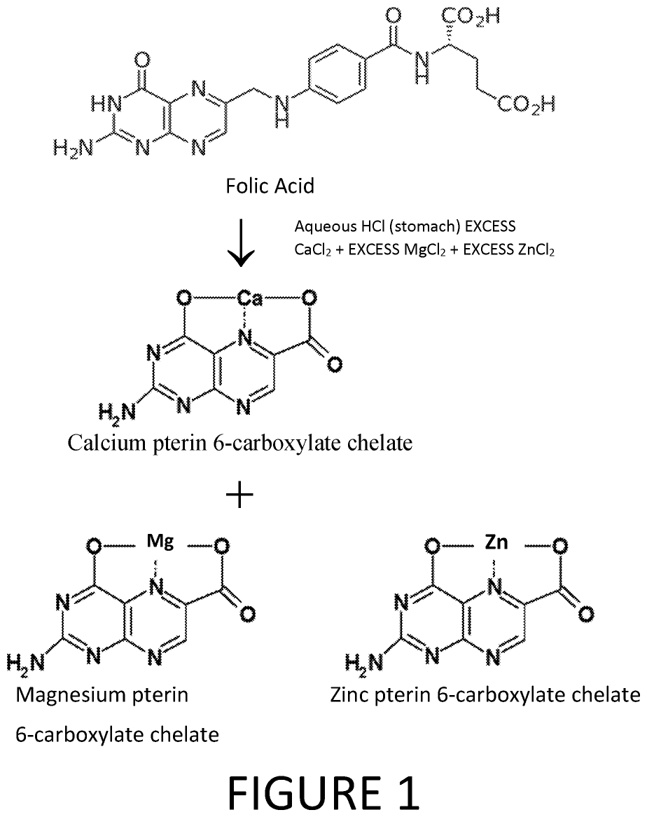

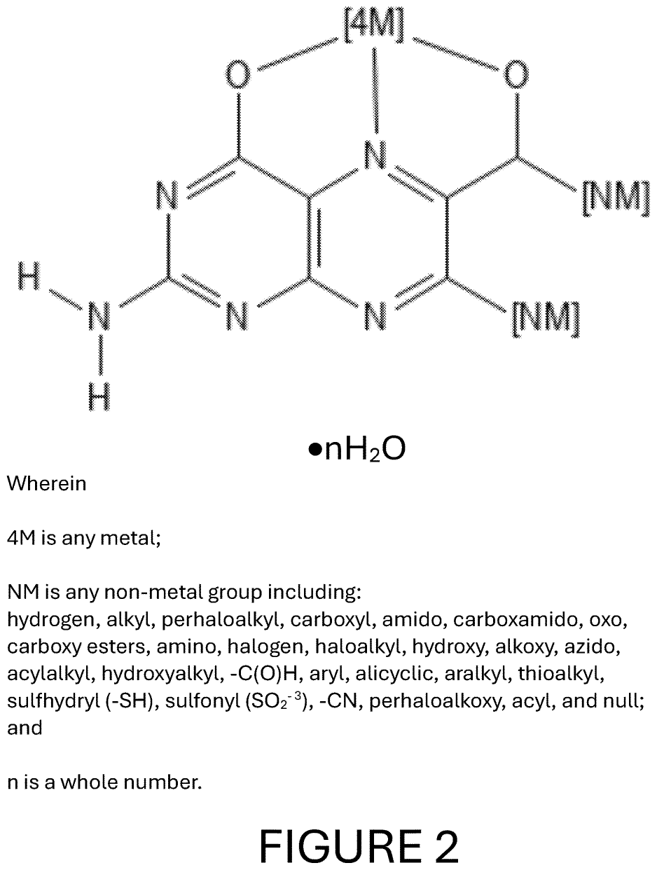

FIG. 2 provides additional active modifications of the FIG. 1 chemical compositions wherein:
4M is any metal; and
NM is any non-metal group including:
hydrogen, alkyl, perhaloalkyl, carboxyl, amido, carboxamido, oxo, carboxy esters, amino, halogen, haloalkyl, hydroxy, alkoxy, azido, acylalkyl, hydroxyalkyl, —C(O)H, aryl, alicyclic, aralkyl, thioalkyl, sulfhydryl (—SH), sulfonyl (SO₂⁻³), —CN, perhaloalkoxy, acyl, and null.

### DETAILED DESCRIPTION

Reference now will be made in detail to embodiments of the disclosure, one or more examples of which are illustrated in the drawings. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment can be used with another embodiment to yield a still further embodiment. Thus, it is intended that the present disclosure covers such modifications and variations as come within the scope of the appended claims and their equivalents.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

As used herein, including in the claims, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination. Also, as used herein, including in the claims, “or” as used in a list of items (for example, a list of items prefaced by a phrase such as “at least one of” or “one or more of”) indicates an inclusive list such that, for example, a phrase referring to “at least one of” a list of items refers to any combination of those items, including single members. As an example, “at least one of: A, B, or C” is intended to cover A, B, C, A-B, A-C, B-C, and A-B-C, as well as any combination with multiples of the same element (e.g., A-A A-A-A, A-A-B, A-A-C, A-B-B, A-C-C, B-B, B-B-B, B B-C, C-C, and C-C-C or any other ordering of A, B, and C).

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

Unless otherwise defined or implied herein, all terms (including technical and scientific terminology) used in this document retain their commonly understood meanings as recognized by those skilled in the relevant field. Furthermore, it is to be understood that terms, including those defined in standard dictionaries, should be interpreted in a manner that is consistent with their usage within the context of the relevant field and this disclosure. These terms should not be construed in an idealized or overly formal sense unless explicitly specified otherwise herein.

A new chemical moiety identified to be ImmunoFolate (IF), which has the components of folic acid, calcium chloride, magnesium chloride has been found to exhibit potent therapeutics effects including anti-inflammatory, anti-viral, and antineoplastic properties. The ImmunoFolate formulation can also include Vitamin B1. The ImmunoFolate formulation can also include Vitamin B12. The ImmunoFolate formulation can also include other vitamins as well. ImmunoFolate can be used as a treatment against Covid, the common colds, and the flu along with numerous other similar ailments. The ImmunoFolate formulation can act as an Antiviral and address afflictions such as hepatitis B virus (HBV). The ImmunoFolate formulation can act as an Anti-mycoplasma (Tuberculosis). The ImmunoFolate formulation can act as an Anti-Diabetes I agent and an Anti-Diabetes II. The ImmunoFolate formulation can act as an Adjunctive Psychotherapeutic. The disclosure can further provide a method of treating an inflammatory-based disease or disorder in a subject comprising administration of a composition comprising of ImmunoFolate formulation. The disclosure can further provide a method of treating an inflammatory-based disease or disorder in a subject comprising administration of a composition comprising of ImmunoFolate formulation, wherein the subjects are human. The disclosure can further provide a method of treating an inflammatory-based disease or disorder in a subject comprising administration of a composition comprising of ImmunoFolate formulation, wherein the subjects are canines or felines.

A second new chemical moiety identified to be ImmunoFolate (IF) plus zinc, which has the components of folic acid, calcium chloride, and Zinc, has been found to exhibit potent therapeutics effects including anti-inflammatory anti-viral, and antineoplastic properties. The ImmunoFolate plus zinc formulation can also include Vitamin B1. The ImmunoFolate plus zinc formulation can also include Vitamin B12. The ImmunoFolate plus zinc formulation can also include other vitamins as well. ImmunoFolate plus zinc can be used as a treatment against Covid, the common colds, and the flu along with numerous other similar ailments. The ImmunoFolate plus zinc formulation can act as an Antiviral and address afflictions such as hepatitis B virus (HBV). The ImmunoFolate plus zinc formulation can act as an Anti-mycoplasma (Tuberculosis). The ImmunoFolate plus zinc formulation can act as an Anti-Diabetes I agent and an Anti-Diabetes II. The ImmunoFolate plus zinc formulation can act as an Adjunctive Psychotherapeutic. The disclosure can further provide a method of treating an inflammatory-based disease or disorder in a subject comprising administration of a composition comprising of ImmunoFolate plus zinc formulation, wherein the subjects are human. The disclosure can further provide a method of treating an inflammatory-based disease or disorder in a subject comprising administration of a composition comprising of ImmunoFolate plus zinc formulation, wherein the subjects are canines or felines.

Both ImmunoFolate and ImmunoFolate plus zinc can be utilized to treat inflammatory-based disease or disorder is arthritis. Arthritis could mean osteoarthritis, rheumatoid arthritis, gout, psoriatic arthritis, lupus, or septic arthritis.

The optimum daily dosing of the chemical composition is described using “μg” to represent micrograms and “mg” to represent milligrams.

The optimum daily dosing of ImmunoFolate is as follows:
Folic Acid 600 μg
CaCl2 (plus hydrates thereof) 50 mg
MgCl2 (plus hydrates thereof) 100 mg
ZnSO4 (plus hydrates thereof) 8 mg; and
Vitamin B12 25 μg

The optimum daily dosing of ImmunoFolate without Zinc is as follows:
Folic Acid 600 μg
CaCl2 (plus hydrates thereof) 50 mg
MgCl2 (plus hydrates thereof) 100 mg
Vitamin B12 25 μg

The optimum daily dosing of ImmunoFolate without Zinc and Magnesium is as follows:
Folic Acid 600 μg
CaCl2 (plus hydrates thereof) 50 mg
Vitamin B12 25 μg

Other effective dosing of ImmunoFolate includes a daily dose of the following:
Folic Acid 200-1000 μg
CaCl2 (plus hydrates thereof) 19-106 mg
MgCl2 (plus hydrates thereof) 25 mg-1,000 mg
ZnSO4 (plus hydrates thereof) 2-10 mg
Vitamin B12 10-100 μg
Vitamin D3-5,000 IU
Multivitamins
Taurine-1,000 mg
Alpha Lipoic Acid-600 mg-1200 mg
Vitamin K2 100 μg
GlyNAC [Glycine 1,000 mg+N-Acetyl L-Cysteine 100 mg] [or GlyNACET [Glycine 1,000 mg+N-Acetyl L-Cysteine Ethyl Ester 100 mg]]

The optimum daily dosing of ImmunoFolate is as follows
Folic Acid 200-1000 μg
CaCl2 (plus hydrates thereof) 19-106 mg
MgCl2 (plus hydrates thereof) 25-1,000 mg
ZnSO4 (plus hydrates thereof) 2-10 mg

Other effective dosing of ImmunoFolate is as follows includes a daily dose of the following:
Folic Acid 200-1000 μg
CaCl2 (plus hydrates thereof) 19-106 mg
MgCl2 (plus hydrates thereof) 25-1,000 mg
ZnSO4 (plus hydrates thereof) 2-10 mg

### SUMMARY OF THE DISCLOSURE

The present disclosure is directed to novel metal Folic Acid complexes of the formula:

Y(MXₐ•b[H₂O]):(Folic Acid)

wherein
M is a bivalent metal ion selected from the group consisting of Ca²⁺, Cu²⁺, Mg²⁺, V²⁺, Cr²⁺, Mn²⁺, Fe²⁺, Co²⁺, Zn²⁺, Mo²⁺, Sr²⁺, Ba²⁺, Ra²⁺, Ru²⁺, Rh²⁺, Pd²⁺, Cd²⁺, Sn²⁺, W²⁺, Re²⁺, Os²⁺, Ir²⁺, Pt²⁺, Si²⁺, and Sm²⁺;
M is a metal ion of charge 2+ or greater;
X is an anion of an acid and has a charge of −1 or less when ionized;
a is an integer of from 1 or greater;
b is an integer for all hydrates; and
Y is an integer of from 3 or greater.

Below is Table 1 which provides a complete blood count (cbc) with different measurements wherein the symbol “uL” represents microliters.

TABLE 1
COMPLETE BLOOD COUNT (CBC) DIFFERENT MEASURES
[Standard Ranges]

| | May 11, 2018 | Feb. 28, 2023 | Mar. 28, 2023 |
|---|---|---|---|
| | While dosing | while dosing | while dosing |
| | With IP | with IF | with IF |
| Basophils (Absolute) | 0.063 | 0.1 | 0.07 |
| [0.0-0.2 × 10E3/uL] | | | |
| Eosinophils (Absolute) | 0.032 | 0.1 | 0.17 |
| [[0.0-0.4 × 10E3/uL] | | | |
| Lymphocytes (Absolute) | 1.327 | 2.1 | 2.46 |
| [0.7-3.1 × 10E3/uL] | | | |
| Monocytes (Absolute) | 0.616 | 0.9 | 0.78 |
| [0.1-0.9 × 10E3/uL] | | | |
| Neutrophils (Absolute) | 5.862 | 6.0 | 4.57 |
| [1.4-7.0 × 10E3/uL] | | | |
| Basophils (%) | 0.8 | 1 | 0.09 |
| [Not Established] | | | |
| Eosinophils (%) | 0.4 | 1 | 2.1 |
| [Not Established] | | | |
| Lymphocytes (%) | 16.8 | 23 | 30.4 |
| [Not Established] | | | |
| Monocytes (%) | 7.8 | 10 | 9.7 |
| [Not Established] | | | |
| White Blood Cells (WBC) | 7.9 | 9.2 | 8.08 |
| [3.4-10.8 × 10E3/uL] | | | |
| Hematocrit (%) | 45.2 | 45.5 | 47.3 |
| [37.5-51.0%] | | | |
| Hemoglobin | 15.6 | 14.9 | 14.8 |
| [13.0-17.7 g/dL] | | | |
| Mean Corpuscular | 30.8 | 29.1 | 29.5 |
| Hemoglobin | | | |
| [26.6-33.0 pg] | | | |
| Mean Corpuscular | 34.5 | 32.7 | 31.3 |
| Hemoglobin | | | |
| Concentration | | | |
| [31.5-35.7 g/dL] | | | |
| Mean Corpuscular Volume | 89.2 | 89 | 94.2 |
| [79-97 fL] | | | |
| Platelets | 300 | 265 | 308 |
| [150-450 × 10E3/uL] | | | |
| Red Blood Cells | 5.07 | 5.12 | 5.02 |
| [4.14-5.80 × 10E3/uL] | | | |
| Red Cell Distribution | 13.1 | 13.2 | 15.0 |
| Width (%) | | | |
| [11.6-15.4%] | | | |

Below is Table 2 which provides a comprehensive metabolic panel with different measurements.

TABLE 2
COMPREHENSIVE METABOLIC PANEL, BLOOD
[Standard Range]

| | Jan. 3, 2022 | | Feb. 28, 2023 | | Mar. 28, 2023 |
|---|---|---|---|---|---|
| | While dosing | | while dosing | | while dosing |
| | With IP | | with IF | | with IF |
| Total Protein and Albumin/Globulin | 1.7 | | 1.8 | | — |
| Ratio (A/G) | | | | | |
| [1.2-2.2] | | | | | |
| Albumin | 4.7 | | 4.7 | | 4.4 |
| [3.8-4.8 g/dL] | | | | | |
| Alkaline Phosphatase | 65 | | 63 | | — |
| [44-121 IU/L] | | | | | |
| Alanine Transaminase (aka SGPT Test) | 18 | | 31 | | — |
| [0-44 IU/L] | | | | | |
| Aspartate Aminotransferase | 14 | | 23 | | — |
| [0-40 IU/L] | | | | | |
| Bilirubin, Total | 0.3 | | <0.2 | | — |
| [0.0-1.2 mg/dL] | | | | | |
| Blood Urea Nitrogen (BUN) | 23 | | 22 | | 21 |
| [0-27 mg/dL] | | | | | |
| BUN/Creatinine Ratio | 14 | | 15 | | 17 |
| [10-24] | | | | | |
| Calcium | 10.2 | | 9.9 | | 9.9 |
| [8.6-10.2 mg/dL] | | | | | |
| Chloride | 106 | | 103 | | 104 |
| [96-106 mmol/L] | | | | | |
| Carbon Dioxide | 27 | | 23 | | 29 |
| [20-29 mmol/L] | | | | | |
| Creatinine | 1.66 | H | 1.50 | H | 1.20 |
| [0.76-1.27 mg/dL] | | | | | |
| eGFR (estimated glomerular | 41 | L | 50 | L | 59.9 |

| filtration rate) | | | | |
|---|---|---|---|---|
| [>59 mL/min/1.73] | | | | |
| Globulin, Total | 2.7 | 2.6 | | — |
| [1.5-4.5 g/dL] | | | | |
| Glucose | 99 | 100 | H | 96 |

| [70-99 mg/dL] | | | | |
|---|---|---|---|---|
| Potassium | 5.3 | H | 4.3 | 5.2 |
| [3.5-5.2 mmol/L] | | | | |
| Sodium | 146 | H | 141 | 141.0 |

| | | | |
|---|---|---|---|
| [134-144 mmol/L] | | | |
| Protein, Total, Serum | 7.4 | 7.3 | — |
| [6.0-8.5 g/dL] | | | |

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating a subject for a disease or disorder comprising
cancer,
an inflammatory-based disease or disorder,
arthritis,
osteoarthritis,
rheumatoid arthritis,
gout,
psoriatic arthritis,
lupus, or
septic arthritis,
a viral infection,
mycoplasma,
diabetes type one,
diabetes type two,
chronic kidney disease, based upon blood creatinine levels, comprising:
administration of a composition of ImmunoFolate which comprises:
folic acid,
calcium chloride (CaCl₂)) or hydrates thereof or both, and
magnesium chloride (MgCl₂) or hydrates thereof or both, and
optionally Vitamin B12;
dosing daily in the following ranges
Folic acid: at least 200 μg,
CaCl₂): at least 19 mg,
MgCl₂: at least 25 mg, and
optionally Vitamin B12: at least 10 μg.

2. The method of claim 1, wherein ImmunoFolate further comprises zinc as zinc sulfate (ZnSO₄) or hydrates thereof or both.

3. The method of claim 1, wherein ImmunoFolate further comprises Vitamin B1.

4. The method of claim 1, wherein ImmunoFolate further comprises any combination of Vitamin D3, Vitamin K2, an amino acid, taurine, an antioxidant, alpha-lipoic acid, GlyNAC.

5. The method of claim 1, wherein said dosing daily comprises dosing in the following ranges: ZnSO₄: 2-10 mg.

6. The method of claim 1, wherein said dosing daily comprises dosing in the following amounts:
Folic acid: 800 μg,
CaCl₂): 106 mg,
MgCl₂: 500 mg, and
optionally Vitamin B12: 25 μg.

7. The method of claim 1, wherein said dosing daily comprises dosing at:
ZnSO₄: 8 mg.

8. The method of claim 1, wherein said dosing daily comprises dosing in the following amounts:
Folic acid: 600 μg,
CaCl₂): 50 mg,
MgCl₂: 100 mg.

9. The method of claim 1, wherein the ImmunoFolate is administered through oral, parenteral, intravenous, subcutaneous, intrathecal, intramuscular, buccal, intranasal, epidural sublingual, pulmonary, local, rectal, or transdermal administration.

10. The method of claim 1, wherein the subject is human, canine or feline.
